(19) United States
(12) Patent Application Publication
(10) Pub. No.: US 2010/0297753 A1
(43) Pub. Date: Nov. 25, 2010

(54) TRANSFECTION BY LASER PORATION ON ROTATING CYLINDER

(76) Inventor: Charles W. Ragsdale, Concord, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/522,254

(22) PCT Filed: Apr. 13, 2009
(86) PCT No.: PCT/US09/40371

§ 371 Date: Sep. 14, 2009

(30) Foreign Application Priority Data
Apr. 15, 2008 (US) 61045137

(51) Int. Cl. C12N 5/10; C12M 1/42

(52) U.S. Cl. 435/325; 435/283.1

(57) ABSTRACT

Biological cells are transfected by a laser poration process in which the structures are immobilized on a solid surface that is cylindrical in shape, and the cylinder, while immersed in a liquid solution of the transfecting species or otherwise in contact with the solution, is rotated past a stationary laser such that the laser beam spans the entire circumference of the cylinder and all cells immobilized thereon. Axial movement of the cylinder or the laser in addition to the rotational movement brings the entire length of the cylinder within the influence of the laser as well as the cylinder circumference.

### CROSS-REFERENCE TO RELATED APPLICATION

This application claims the benefit of U.S. Provisional Patent Application No. 61/045,137, filed Apr. 15, 2008, the contents of which are incorporated herein by reference.

### BACKGROUND OF THE INVENTION

1. Field of the Invention

This invention lies in the field of transfection, the process by which exogenous molecular species are inserted into membranous structures by rendering the membrane permeable on a transient basis while the structure is in contact with a liquid solution of the species, thereby allowing the species to pass through the membrane, and doing so in such a manner that the structure resumes its viability after the completion of the procedure. In particular, this invention relates to methods of transfection that utilize light energy to achieve the transient permeabilization.

2. Description of the Prior Art

The introduction of exogenous species, including hydrophilic or membrane-impermeant species, into biological cells is of use in certain biologic and biochemical techniques. A high efficiency transfection is one in which the exogenous species has entered a high proportion of the cells while the viability of the cells has either been maintained-throughout or restored after the procedure. Of the various transfection techniques, electroporation, which is the use of an electric field as the source of energy for the membrane permeabilization, has received the most attention. Electroporation suffers from low efficiency, however, and such interfering factors as the production of gas at the electrode surface.

Optical energy has been found be a viable substitute for electrical energy, and has led to studies involving the use of lasers as the energy source. The resulting procedures have variously been termed laser poration, optoinjection, optoporation, and photoporation, and disclosures of these procedures are found in both the patent literature and technical journals. Examples of these disclosures are: Lemelson, U.S. Pat. No. 5,795,755 (issued Aug. 18, 1998); Koller et al., U.S. Pat. No. 6,753,161 B2 (issued Jun. 22, 2004); Koller et al., United States Patent Application Publication No. US 2005/0095578 A1 (published May 5, 2005); Koller et al., U.S. Pat. No. 7,300,795 B2 (issued Nov. 27, 2007); Dholakia et al. (University of St. Andrews), International Patent Application No. WO 2006/059084 A1 (published Jun. 8, 2006); Paterson, L., et al., “Photoporation and cell transfection using a violet diode laser,” Optics Express, vol. 13, no. 2, pp. 595-600 (Jan. 24, 2005); Soughayer, J. S., et al., “Characterization of Cellular Optoporation with Distance,” Anal. Chem. 2000, 72, 1342-1347 (Mar. 15, 2000); and Kohli, V., et al., “An alternative method for delivering exogenous material into developing zebrafish embryos,” Biotechnology and Bioengineering, vol. 98, issue 6, pages 1230-1241 (Jul. 5, 2007).

While the terms “laser poration” and “photoporation” are generic to the use of a laser for permeabilization of a membrane structure, the terms optoinjection and optoporation have relatively focused meanings. “Optoinjection” refers to the irradiation of a single cell (or other membranous structure) by a focused laser beam to achieve transfection of that cell alone, whereas “optoporation” refers to directing the laser beam to a continuous absorptive medium in which the cells are suspended to generate a mechanical transient or stress wave in the suspending medium which is transmitted to the cells through the medium.

Of further potential relevance to this invention is transfection as applied to adherent cells. One system for laser poration of adherent cells is disclosed by Iwata et al. in United States Patent Application Publication No. US 2007/0059832 A1 (published Mar. 15, 2007).

### SUMMARY OF THE INVENTION

The present invention resides in apparatus and method for performing optoinjection by moving an array of adherent biological cells relative to the laser beam in a controlled and highly efficient manner to cause a large number of cells to be transiently permeabilized in succession and to provide each cell will have a high probability of successful transfection.

The adherent cells in the practice of this invention are immobilized on a surface of a cylinder toward which a laser beam is directed, and the cylinder is rotated past the laser beam to cause the adherent cells to pass through the path of the laser beam so that eventually all, or substantially all, of the cells are exposed to the beam. The cylinder is a circular cylinder with a uniform diameter along its axis, solid in certain embodiments of the invention and hollow in others. With a hollow cylinder, the cells can either be on the inner, concave surface of the cylinder or the outer, convex surface, while with a solid cylinder, the cells are on the outer, convex surface. Likewise, with a hollow cylinder, the laser can either be positioned inside the cylinder or outside the cylinder, while with a solid cylinder, the laser is positioned outside the cylinder. The surface of the cylinder on which the cells are immobilized is of uniform diameter to place all cells at substantially the same distance from the laser as the cells enter the laser beam while the cylinder is rotated about its axis. Regardless of the Position of the laser relative to that of the cylinder, the laser does not rotate with the cylinder, and in preferred embodiments of the invention the laser remains stationary as the cylinder rotates. Either a single laser or an array of lasers, such as a linear row of lasers parallel to the axis of the cylinder, can be used. In certain embodiments as well, rotation of the cylinder about its axis is accompanied by linear axial movement of the cylinder, particularly when a single laser is used or when an array of lasers is used that does not extend the entire length of the cylinder. During the rotation of the cylinder, the cells entering the laser beam are maintained in contact with the buffer solution in which the species to be inserted in the cells is dissolved. Such contact is achieved either by total immersion of the cylinder in the solution when the cells are on the outside of the cylinder, filling the cylinder with the solution when the cells are on the inside of the cylinder, by placing a film of the solution over the cylinder surface or at least the portion that is in the path of the laser beam, or by causing a film of the solution to cascade over the cylinder surface. The cylinder itself can serve as a reservoir of the solution, or can be immersed in a reservoir of the solution, or a reservoir that is separate from the cylinder can be used, providing a continuous feed of the solution to the cylinder or the cylinder surface.

A more detailed understanding of these and other features, objects, advantages, and embodiments of the invention will be gained from the descriptions that follow.

### BRIEF DESCRIPTION OF THE DRAWINGS

FIG. 1 is a perspective view of a laser poration apparatus in accordance with the present invention.

FIGS. 2a and 2b are views of the interior of the laser poration apparatus of FIG. 1 in two positions, respectively.

FIG. 3 is a perspective view of a second laser poration apparatus in accordance with the present invention.

FIG. 4 is a perspective view of a third laser poration apparatus in accordance with the present invention.

FIG. 5a is a perspective view of a fourth laser poration apparatus in accordance with the present invention. FIG. 5b is an enlarged view in cross section of a portion of the apparatus of FIG. 5a.

FIG. 6 is an enlarged view in cross section of a portion of a fifth laser poration apparatus in accordance with the present invention.

FIG. 7 is a perspective view of a sixth laser poration apparatus in accordance with the present invention.

FIG. 8 is a perspective view of a seventh laser poration apparatus in accordance with the present invention.

FIG. 9 is a perspective view of an eighth laser poration apparatus in accordance with the present invention.

### DETAILED DESCRIPTION OF THE INVENTION AND PREFERRED EMBODIMENTS

Biological cells that can be transfected by the present invention include those that are grown on the surface on which they are to be transfected and are naturally adherent thereto and those whose adherence is enhanced by cell-adhesive molecules that are either coupled to the cells or to the surface. All such cells are referred to herein as “adherent cells.” Examples of adherent cells are neuronal cells, neuronal stem cells, mesenchymal stem cells, pancreatic cells, skeletal muscle cells, cardiomyocytes, and liver or liver-derived cells such as primary hepatocytes, liver epithielial cells, HepG2 cells, and hepatocellular carcinoma-derived cells. Examples of the species to be inserted into the cells by the present invention are nucleic acids including DNA, RNA, plasmids, and genes and gene fragments, as well as proteins, pharmaceuticals, and enzyme cofactors. Further examples will be apparent to those skilled in the art.

Solid surfaces to which the cells will adhere and that will therefore be useful as the cylindrical surface in the practice of this invention will be the surfaces of bodies constructed of any material that is capable of serving as an immobilizing support for the cells. Such a body is preferably a rigid body but can also be a covering over the surface of a rigid body, such as a flexible sheet, a membrane, or a lamina, either continuous, perforated, or of mesh construction. Examples of the surface materials are glass, polycarbonate, polystyrene, polyvinyl, polyethylene, and polypropylene. Examples of flexible surface coverings are microporous membranes used in membrane-based cell culture, such as membranes of hydrophilic poly(tetrafluoroethylene), cellulose esters, polycarbonate, and polyethylene terephthalate. A flexible membrane can be formed into a cylinder by placing the membrane over a cylindrical support such as a cylindrical screen or a cylinder of solid glass or polymeric material. Adherent cells can be immobilized on the surface of a rigid body or a membrane supported by a rigid substrate by conventional means, including the inherent adherence when the cells are grown on the surface, as well as adherence through immunological or affinity-type binding, electrostatic attraction, and covalent coupling.

The dimensions of the cylinder, and particularly of the cylindrical surface on which the adherent cells are immobilized, can vary, although certain considerations may affect the optimal choice for particular systems. For embodiments of the invention in which the cells are on the outside, i.e., the convex surface, of the cylinder, it is preferable that the cylinder be of sufficiently large diameter that the perspective of the cells is substantially the same as that of cells on a flat surface. The cells will then have substantially the same access to each other for signal exchange that they would have on a flat surface. This will avoid disruption or interference with cell functions such as those entailed in differentiating phenotypes among the cells of a cell colony. The minimum cylinder diameter that will produce this effect will vary with the cells, but in most cases, best results will be achieved with a cylinder surface diameter of from about 1 cm to about 30 cm, preferably from about 2 cm to about 20 cm, and most preferably from about 5 cm to about 10 cm. The length of the cylinder is primarily governed by the number of cells that are sought to be transfected, and can vary widely. In most cases, the lengths are contemplated to be from about 0.5 times the diameter to about 5 times the diameter.

The cylinder is rotated about its axis while its axis is either horizontal or vertical, depending on the manner in which contact between the surface and the liquid solution of the transfecting species is maintained. Rotation is achieved by conventional means, such as by stepper motors, dc motors, and manual drives. The rotational speed of the cylinder is limited only by such considerations as avoiding loss of the cells or of the buffer solution and the species dissolved in it due to centrifugal force, and minimizing splashing of the buffer solution or the formation of air bubbles in the solution, both of which will lower the degree of contact of the solution with the cells. In most cases, effective results will be achieved with a rotation speed of from about 10 revolutions per minute (rpm) to about 100 rpm, and preferably from about 20 rpm to about 60 rpm. Preferably, the rotational speed of the cylinder and the axial movement, if any, are selected such that each cell receives multiple passes of the laser, thereby ensuring a high rate of transfection together with high control over the uniformity of the exposure of the cells to the optical energy.

The laser or array of lasers can be positioned either inside the cylinder, in the case of a hollow cylinder, or outside the cylinder, in cases of both hollow and solid cylinders. The term “solid cylinder” is used herein to denote a cylinder that is not hollow and thereby lacks an inner, concave surface. When a hollow cylinder is used, the laser or laser array can be on the same side of the cylinder wall as the adherent cells, or on a side of the wall that is opposite the side with the cells. When the laser(s) and the cells are on opposite sides of the wall, the wall can be made to allow laser beam(s) to penetrate the cylinder wall to reach the cells. Such penetration can be achieved by use of a transparent, or at least light-transmissive, wall or by a perforated wall. Light transmissivity can be achieved by appropriate selection of the wall material or by the use of a thin wall, or both.

Any lasers known in the literature for use in laser poration can be used. Examples are a continuous-wave argon laser (488 nm), pulsed Nd:YAG lasers (1064 nm, 355 nm, or 532 nm), and pulsed, near-infrared titanium-sapphire lasers. The beam diameter at the cell surface will in most cases be within the range of about 0.5 micron to about 100 microns, and preferably from about 1 micron to about 30 microns. For pulsed lasers, the pulse duration will typically be within the range of about 1 femtosecond to about 10 milliseconds, preferably from about 1 nanosecond to about 1 microsecond.

To achieve transfection of cells distributed not only around the circumference of a cylindrical surface but also along its length with a single laser, the laser can be mounted to a carriage that travels in the axial direction of the cylinder. Alternatively, the laser can be stationary and the cylindrical surface itself can travel along its axis. In either case, drive mechanisms of the same types (listed above) used to rotate the cylinder can be used. The axial movement can be coordinated with the cylinder rotation to achieve coverage of the entire population of cells on the surface.

While the features defining this invention are capable of implementation in a variety of constructions and procedures, the invention as a whole will be best understood by a detailed examination of certain specific embodiments such as those shown in the drawings.

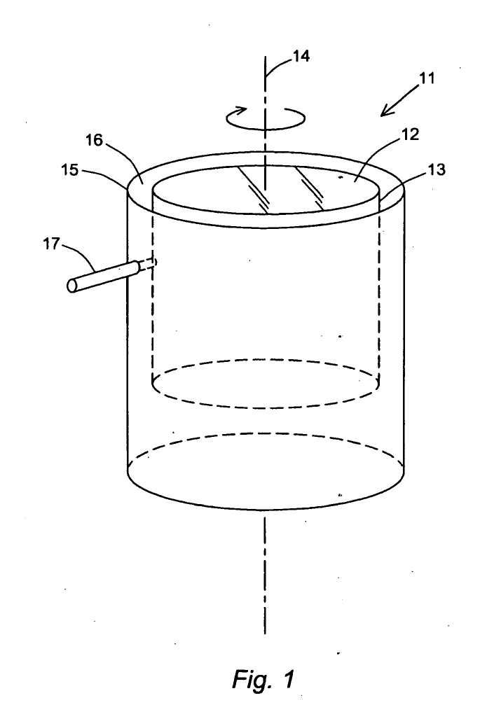

FIG. 1 is a representation of a laser poration apparatus 11 in which the rotating cylinder 12 is a solid cylinder and the cells are adhered to the outer, convex surface 13 of the cylinder. The cylinder 12 rotates about its axis 14 within an outer cylinder 15 that remains stationary as the inner cylinder 12 rotates. The solution of the transfecting species is retained in the annular space 16 between the two cylinders. A single laser 17 is mounted in the wall of the outer cylinder 15 and directs a laser beam through the solution in the annular space 16 to the cells on the outer surface 13 of the inner cylinder 12. With the laser in a fixed position on the outer cylinder 15, the laser beam remains stationary as the inner cylinder rotates and the beam strikes cells around the entire circumference of the inner cylinder.

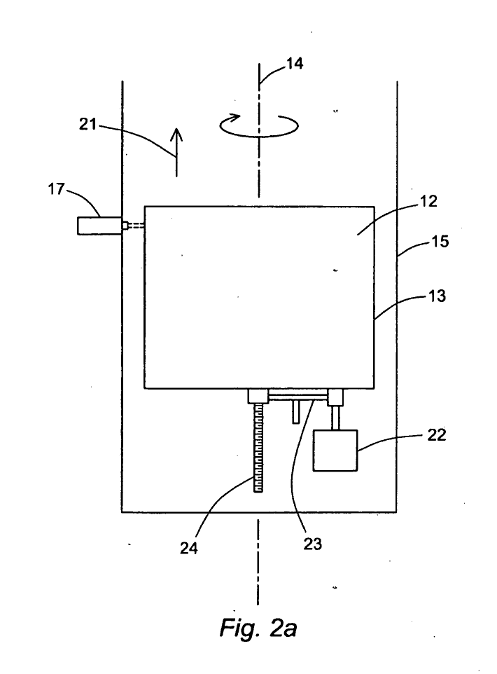

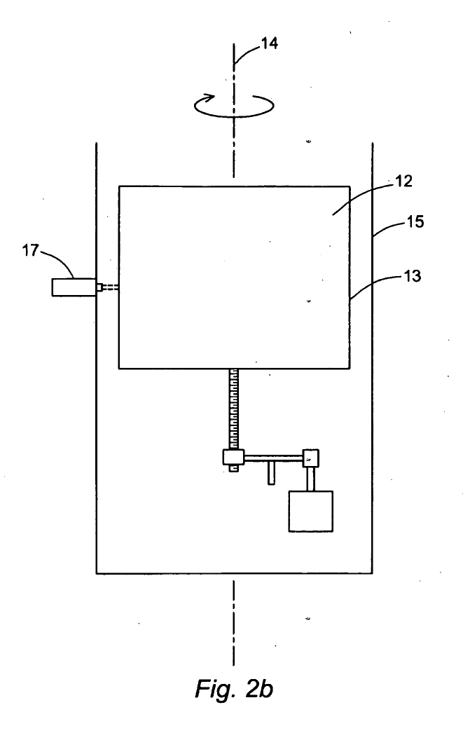

Axial movement of the inner cylinder, which occurs concurrently with the rotation, is illustrated in FIGS. 2a and 2b, which show two positions along the axial travel path, respectively. FIG. 2a shows the direction 21 of the axial movement. In the example shown in FIGS. 2a and 2b, a single motor 22 drives both movements, utilizing a gear train 23 for the rotation and a threaded shaft 24 extending from the bottom of the inner cylinder 12 for the axial movement. Alternatives to the threaded shaft for obtaining axial movement of the cylinder are a magnetic drive, a drive based on electric coils, and any other conventional linear driving mechanism. Whether a threaded shaft or other component is used, it is preferred that it be detachable from the cylinder to facilitate the removal of the cylinder from the mechanism in order to place the cylinder in an incubation chamber where cells can grow on the cylinder surface while exposed to a nutrient medium, gases, or any other species or materials needed to promote the growth. Added efficiency can be achieved by growing cells on one cylinder while cells already grown on a different cylinder are placed in the apparatus for laser poration. A readily detachable coupling such as a magnetic coupling can be used to join the threaded shaft or other mechanism to the cylinder. As an alternative to a cylinder that moves axially, the laser can be made to travel in the axial direction by mounting the laser on a carriage with a linear carriage drive.

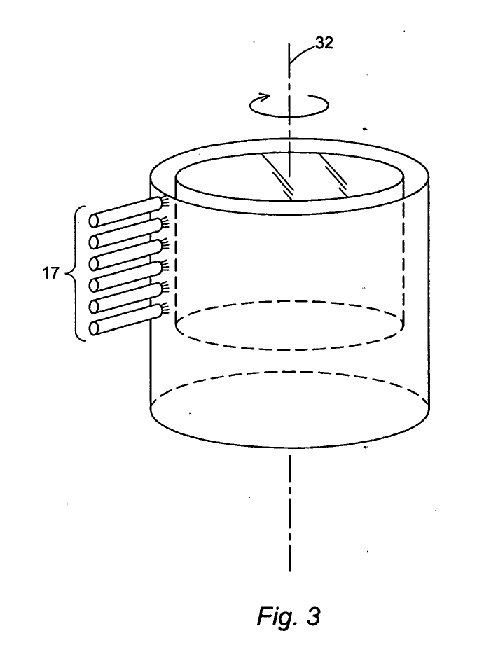

An alternative to axial movement of either the cylinder or the laser is the use of a row of lasers or a laser line light. An example is shown in FIG. 3, where the single laser 17 of FIG. 1 is replaced by a row of lasers 31 arranged in a straight line parallel to the common axis 32 of the two cylinders.

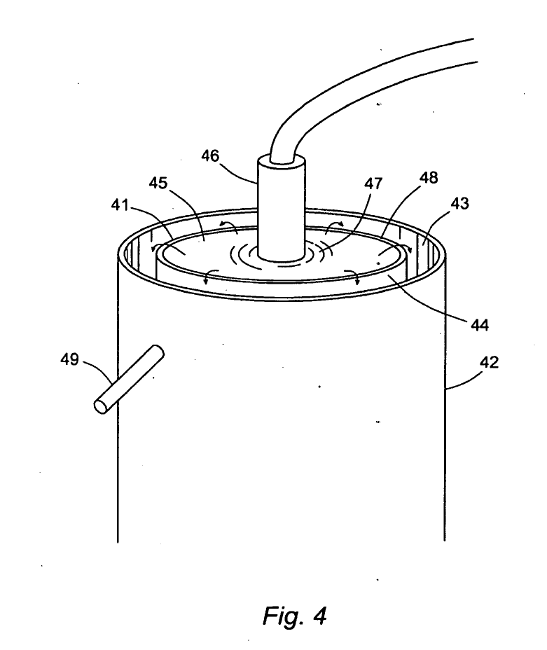

An example of an apparatus using a hollow inner cylinder is shown in FIG. 4. As in FIGS. 1, 2a, and 2b, the inner cylinder 41 rotates within a non-rotating outer cylinder 42, with the two cylinders separated by an annular space 43. The cells adhere to the outer surface 44 of the inner hollow cylinder 41. Unlike FIGS. 2a and 2b in which the cells are immersed in the liquid solution, the liquid solution in FIG. 4 forms a falling film over the cells on the outer surface 44 of the rotating cylinder. The film in this case is formed by filling the interior 45 of the inner cylinder 41 with the solution and continuously adding additional solution to the inner cylinder through a length of tubing 46 whose open end 47 terminates at the upper surface of the liquid. The solution flows over the rim 48 of the inner cylinder 41 to cascade down the outer surface 44 of the cylinder. The cascade continues as the inner cylinder 41 rotates, and the beam from the laser 49 which is mounted to the wall of the outer cylinder 42 strikes the cells around the circumference of the inner cylinder 41. Axial movement of the inner cylinder 41 can be achieved in the same manner as the axial movement illustrated in FIGS. 2a and 2b.

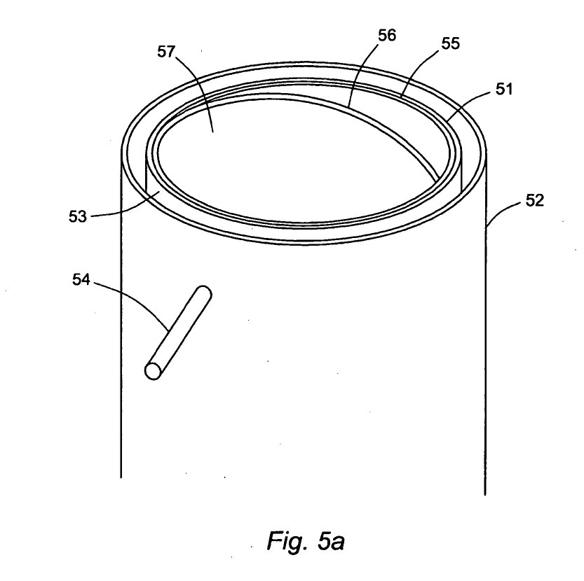

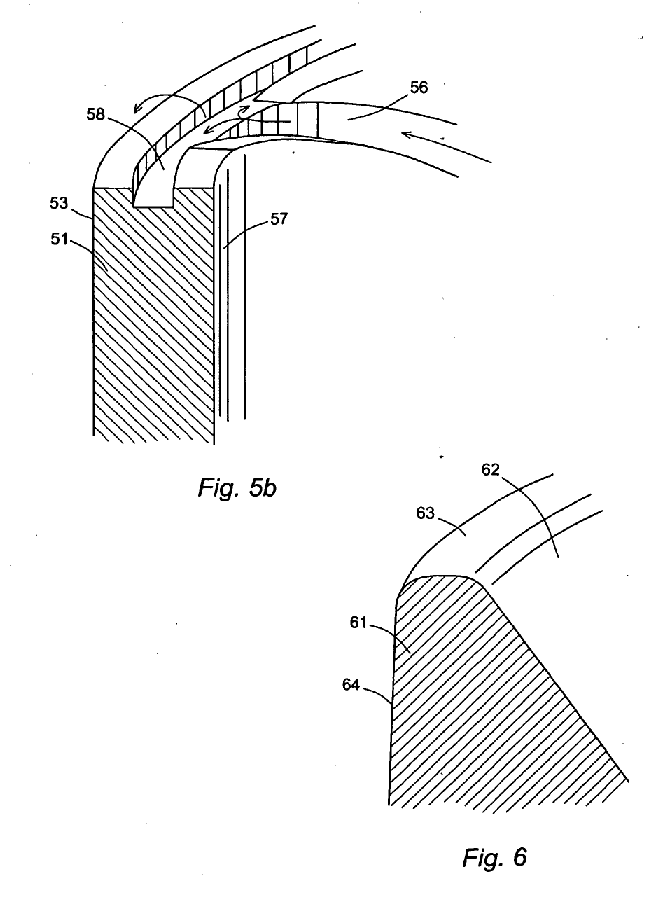

FIGS. 5a and 5b illustrate an alternative structure for forming a falling film on the outer surface of the inner cylinder. The apparatus of FIGS. 5a and 5b is similar to that of FIG. 4 by including an inner cylinder 51 that rotates within a stationary outer cylinder 52. The cells are adhered to the outer surface 53 of the inner cylinder 51 and the laser 54 is mounted within the wall of the outer cylinder 52. The inner cylinder 51 is hollow as in FIG. 4, and serves as a reservoir for the liquid solution of the transfecting species. A falling film of the liquid solution passes from the hollow interior of the inner cylinder over the rim 55 of the cylinder to form a liquid film cascading over the outer surface of the inner cylinder and the adherent cells on the surface. In this embodiment of the invention, however, the liquid solution is drawn up to the rim of the inner cylinder by the rotational movement of the inner cylinder. A spiral groove 56 formed in the inner surface 57 of the cylinder collects the liquid as the cylinder rotates, and the rotational movement causes the liquid to flow up the spiral groove to the cylinder rim. At the rim, the spiral groove 56 opens into a trough 58 in the rim (FIG. 5b), and the trough serves to distribute the liquid around the circumference of the cylinder before flowing over the edge of the rim to form a falling film over the outer surface of the cylinder. While a single spiral groove 56 is shown, the cylinder can contain two or more spiral grooves running parallel for a more uniform distribution of the liquid solution around the rim. Unlike the system of FIG. 4, this system does not require that the interior of the inner cylinder be filled to the rim with the liquid solution.

A still further alternative for forming a continuous falling film over the outer surface of the inner cylinder is illustrated in FIG. 6. In this embodiment, the inner cylinder is again a hollow cylinder 61 but the inner surface 62 of the cylinder is tapered such that rotation of the cylinder will drive the liquid up the inner wall due to the angle of the surface to the cylinder axis in conjunction with the centrifugal force generated by the rotation. Once the liquid reaches the rim 63, it flows over the rim to form a falling film over the outer surface 64 as in the embodiments of FIGS. 4, 5a and 5b.

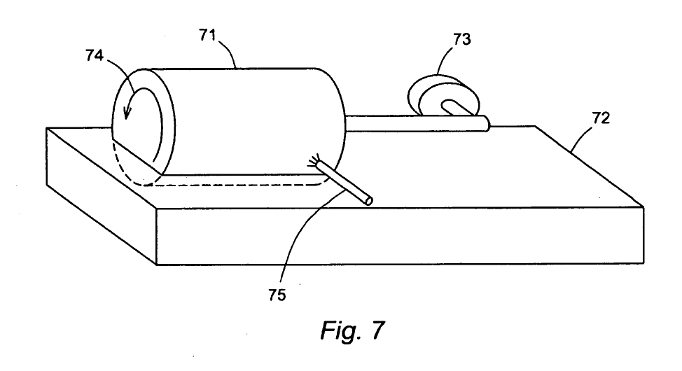

In all embodiments of the invention, the cells must be in contact with the liquid solution during their exposure to the laser beam so that the molecular species dissolved in the liquid will pass through the permeabilized membranes of the cells. Contact with the liquid solution is preferably maintained at all points in the rotation so that the cells will continuously reside in an environment that is favorable to cell viability. In each of the embodiments of FIGS. 4, 5a, 5b, and 6, the falling film serves these functions provided that it does not separate from the cylinder surface and provided that its speed is not so high that its impact causes the cells to separate from the surface. One means of controlling the system to lessen the risk of these occurrences is to place the rotating cylinder in a horizontal orientation in which the cylinder is only partially immersed in the liquid solution. An example of a system that operates in this manner is illustrated in FIG. 7.

The rotating cylinder 71 in FIG. 7 can be either solid or hollow, and the adherent cells are on the outer surface of the cylinder. The cylinder 71 is horizontally oriented and is partially submerged in a tank 72 of the liquid solution. A motor 73 adjacent to the tank causes the cylinder 71 to rotate in the direction indicated by the arrow 74, drawing a film of the liquid solution up over the cylinder surface. A stationary laser 75 is Positioned with its beam aimed at a location just above the liquid surface. In addition to its function of causing the rotation of the cylinder 71, the motor 73 can also be used to move the cylinder along the cylinder axis, allowing the laser beam to span the entire length of the cylinder in addition to its circumference.

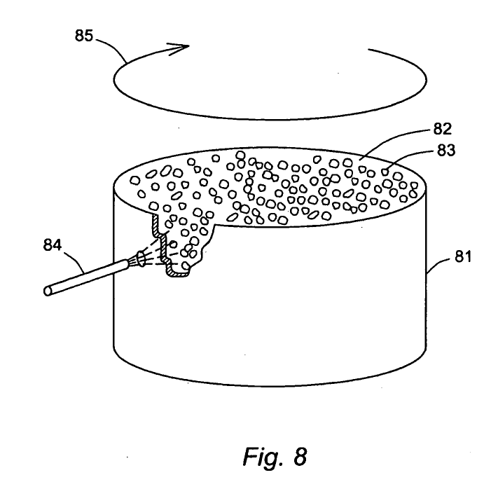

While the foregoing figures represent embodiments in which the cells are immobilized on the outer surface of the rotating cylinder, an alternative that is also within the scope of the invention is to place the cells on the inner surface of a hollow rotating cylinder with the interior of the cylinder filled with the liquid solution. One example of such an arrangement is shown in FIG. 8. The cylinder 81 is shown with a portion of its wall broken away to show the inner surface 82 of the cylinder with the cells 83 immobilized on the inner surface. The liquid solution, although not shown, fills the interior of the cylinder and covers the cells. The laser 84 is outside the cylinder and its beam is directed to the outer surface of the cylinder wall. The wall of the cylinder is sufficiently thin to transmit light energy from the laser beam, so that the light energy penetrates the wall to reach the cells 83 on the inner cylinder surface 82. Rotation of the cylinder in the direction of the arrow 85 occurs as in the embodiments of the Figures discussed above.

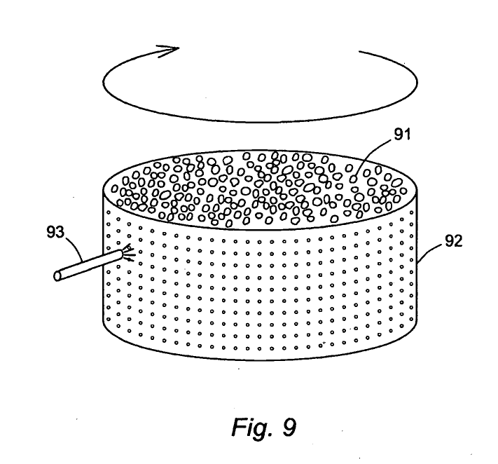

An alternative to the embodiment of FIG. 8 is that of FIG. 9. In this embodiment, similar to that of FIG. 8, the cells 91 are immobilized on the inner surface of the wall of a hollow cylinder 92, and the laser 93 resides outside the cylinder and must penetrate the cylinder wall to reach the cells. This is achieved in the FIG. 9 embodiment by using a cylinder whose wall is perforated with holes through which the laser beam passes when the holes are aligned with the beam. The holes are sized and positioned in such a manner that the laser beam passing through them has a high probability of striking the cells. For cells that are 10 microns to 20 microns in diameter, for example, holes of approximately 4 microns in diameter can be placed at intervals approximately 10 microns apart. A portion of each cell will then reside over one or more holes. A laser that emits light in pulses can be used to advantageous effect, and the timing of the laser pulses can be coordinated with the rotational position of the cylinder. Coordination can be achieved with a set of registration holes distributed around the circumference of the cylinder near the cylinder edge, and an optical device can be used to detect when the registration holes are in positions that correspond to the alignment of the other holes with the laser beam. Other means of position detection known in the art can be used in lieu of the registration holes.

In the claims appended hereto, the term “a” or “an” is intended to mean “one or more.” The term “comprise” and variations thereof such as “comprises” and “comprising,” when preceding the recitation of a step or an element, are intended to mean that the addition of further steps or elements is optional and not excluded. All patents, patent applications, and other published reference materials cited in this specification are hereby incorporated herein by reference in their entirety. Any discrepancy between any reference material cited herein and an explicit teaching of this specification is intended to be resolved in favor of the teaching in this specification. This includes any discrepancy between an art-understood definition of a word or phrase and a definition explicitly provided in this specification of the same word or phrase.

What is claimed is:

1. Apparatus for transfection of adherent biological cells, said apparatus comprising:
a body having a surface to which said cells adhere, said surface forming a right circular cylinder about an axis;
a laser oriented such that a laser beam produced-thereby is intercepted by said surface;
a reservoir in fluid communication with said surface; and
means for rotating said body about said axis to draw said surface across said laser beam.

2. The apparatus of claim 1 wherein said surface is an outer, convex surface of said body.

3. The apparatus of claim 1 wherein said surface is an inner, concave surface of said body.

4. The apparatus of claim 1 further comprising means for causing said laser to travel relative to said body along a path parallel to said axis.

5. The apparatus of claim 1 further comprising means for causing said body to travel relative to said laser along a path parallel to said axis.

6. The apparatus of claim 1 comprising a plurality of said lasers distributed along a direction parallel to said axis.

7. The apparatus of claim 6 wherein said plurality of lasers are arranged in a straight line parallel to said axis.

8. The apparatus of claim 1 wherein said body has a hollow interior terminating at an open top surrounded by a rim, said reservoir is said hollow interior, and said surface is an outer, convex surface of said body, said apparatus further comprising means for causing a liquid solution of transfecting species to flow from said hollow interior over said rim to form a falling film over said outer, convex surface.

9. The apparatus of claim 8 wherein said means for causing said liquid solution to flow over said rim is means for continuously feeding said liquid solution to said hollow interior to cause overflow of said liquid solution from said hollow interior over said rim.

10. The apparatus of claim 8 wherein said means for causing said liquid solution to flow over said rim is a spiral groove in an inner, concave surface of said hollow interior.

11. The apparatus of claim 1 wherein said surface is an outer, convex surface of said body, said reservoir is arranged to retain a liquid of transfecting species with a horizontal liquid surface, and said body is mounted in said apparatus to enable partial submersion of said outer, convex surface of said body below said liquid surface with said axis parallel to said liquid surface.

12. The apparatus of claim 11 wherein said laser is positioned outside of said body, and said body is sufficiently transmissive of light to allow light from said laser to penetrate said body to reach adherent cells on said inner, concave surface.

13. The apparatus of claim 11 wherein said laser is positioned outside of said body, and said body is apertured with an array of holes to allow light from said laser to pass through said holes to reach adherent cells on said inner, concave surface.

14. In a process for the transfection of a population of biological cells comprising exposing said cells to a laser beam while said cells are in contact with a liquid solution of a transfecting species, the improvement comprising:
immobilizing said cells on a surface that forms a right circular cylinder about an axis; and
directing said laser beam from said laser to said surface while rotating said surface about said axis to draw said surface across said laser beam and while contacting said immobilized cells with said liquid solution of transfecting species.

15. The process of claim 14 wherein said surface is an outer, convex surface of a cylindrical body.

16. The process of claim 14 wherein said surface is an inner, concave surface of a cylindrical body.

17. The process of claim 14 further comprising moving said laser beam along a path of travel parallel to said axis while rotating said surface.

18. The process of claim 14 further comprising moving said surface along a path of travel parallel to said axis while rotating said surface.

19. The process of claim 14 comprising directing laser beams from a plurality of lasers to said surface while rotating said surface about said axis, said lasers distributed along a direction parallel to said axis.

20. The process of claim 19 wherein said plurality of lasers are arranged in a straight line parallel to said axis.

21. The process of claim 14 wherein said surface is an outer, convex surface of a cylindrical body with a hollow interior terminating at an open top surrounded by a rim, said process further comprising causing said liquid solution to flow from said hollow interior over said rim to form a falling film over said immobilized cells.

22. The process of claim 21 comprising continuously feeding said liquid solution to said hollow interior to cause said liquid solution to overflow from said hollow interior over said rim.

23. The process of claim 21 wherein said hollow interior has a spiral groove, and rotation of said surface is achieved by rotating said cylindrical body in a direction that causes said liquid solution to travel through said groove to and over said rim.

24. The process of claim 14 wherein said surface is an outer, convex surface of a cylindrical body, said process comprising rotating said cylindrical body with said axis horizontally oriented while said cylindrical body is partially submerged in a layer of said liquid solution to draw a film of said liquid solution over a surface region of said cylindrical body above said layer of liquid solution, and directing said laser beam to a spot on said surface region as said cylindrical body rotates.

25. The process of claim 14 wherein said surface is an inner, concave surface of a cylindrical tube having a wall that is sufficiently transmissive of light from said laser beam to allow said light to penetrate said tube to reach said cells, said process comprising directing said laser beam through said wall of said cylindrical tube.

26. The process of claim 14 wherein said surface is an inner, concave surface of a cylindrical tube having a wall that is apertured with an array of holes, said process comprising directing said laser beam through said holes to reach said cells.
